(19) United States
(12) Patent Application Publication
(10) Pub. No.: US 2025/0032400 A1
(43) Pub. Date: Jan. 30, 2025

(54) PERSONAL CARE COMPOSITION

(71) Applicant: Conopco, Inc., d/b/a UNILEVER, Englewood Cliffs, NJ (US)

(72) Inventors: Xuelan GU, Shanghai (CN); Tingyan MI, Shanghai (CN)

(73) Assignee: Conopco, Inc., d/b/a UNILEVER, Englewood Cliffs, NJ (US)

(21) Appl. No.: 18/716,615

(22) PCT Filed: Dec. 19, 2022
(86) PCT No.: PCT/EP2022/086751

§ 371 Date: Jun. 5, 2024

(30) Foreign Application Priority Data
Dec. 31, 2021 (WO) PCT/CN2021/143516
Jan. 27, 2022 (EP) 22153548.7

(51) Int. Cl. A61K 8/9789; A61K 8/06; A61K 8/46; A61Q 19/00; A61Q 19/08

(52) U.S. Cl. CPC A61K 8/9789; A61K 8/062; A61K 8/46; A61Q 19/007; A61Q 19/08

(57) ABSTRACT

Disclosed is a personal care composition comprising carboxymethyl cysteine compound and Sandalwood extract.

### FIELD OF THE INVENTION

The present invention relates to a personal care composition comprising a carboxymethyl cysteine compound and Sandalwood extract for anti-aging.

### BACKGROUND OF THE INVENTION

Aging is an inevitable progression of life. Consumers seek to alleviate or delay the signs of chronological aged or photoaged skin, such as fine lines and wrinkles, dry and sagging skin. Various strategies have been developed to achieve the anti-aging effects for consumers.

Hyaluronic acid is found in skin not only in the dermis but also in the epidermal intercellular spaces. It can bind and retain huge amount of water and may help moisturize and firm the skin. The content of hyaluronic acid decreases with age, which is one factor believed to account for the change in aged skin, such as loss of skin moisture, as well as the development of wrinkles and fine lines. Therefore, it is desired to boost the synthesis stimulate and the function of hyaluronic acid.

HAS3 (hyaluronan synthase 3) plays an important role in regulating synthesis of hyaluronic acid in the epidermis. The interaction of hyaluronic acid with CD44 cell surface glycoprotein is a driver of collagen synthesis and other hyaluronic acid mediated skin effects, like proliferation of keratinocytes. The synthesis of hyaluronic acid and collagen in the skin as well as other aging processes in the skin may be improved by elevating the expression of HAS3 and CD44. The present inventors have recognized such needs and have developed a personal care composition comprising carboxymethyl cysteine compound and Sandalwood extract. It was surprisingly found that by combining carboxymethyl cysteine compound with Sandalwood extract, the expression of both HAS3 and CD44 are significantly elevated.

### SUMMARY OF THE INVENTION

In a first aspect, the present invention is directed to a personal care composition comprising carboxymethyl cysteine compound and Sandalwood extract.

In a second aspect, the present invention is directed to a method of improving skin moisturization; treating, reducing, and/or preventing of fine lines or wrinkles of skin; improving collagen production in skin; providing anti-aging benefit to skin; and/or firming skin, comprising a step of topically applying to the skin the composition of the present invention.

In a third aspect, the present invention is directed to use of the composition of the present invention for improving skin moisturization; treating, reducing, and/or preventing of fine lines or wrinkles of skin; improving collagen production in skin; providing anti-aging benefit to skin; and/or firming skin.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

The carboxymethyl cysteine compound refers to compound selected from carboxymethyl cysteine, salt of carboxymethyl cysteine, ester of carboxymethyl cysteine, amide of carboxymethyl cysteine or a mixture thereof. Preferably, the carboxymethyl cysteine compound comprises carboxymethyl cysteine, ester of carboxymethyl cysteine, and/or salt of carboxymethyl cysteine. More preferably, the carboxymethyl cysteine compound comprises carboxymethyl cysteine, and/or salt of carboxymethyl cysteine. Even more preferably, carboxymethyl cysteine compound comprises salt of carboxymethyl cysteine. Still even more preferably the carboxymethyl cysteine compound comprises lysine carboxymethyl cysteinate and most preferably, the carboxymethyl cysteine compound is lysine carboxymethyl cysteinate.

Preferably, the carboxymethyl cysteine compound is present in amount of at least 0.00001%, more preferably at least 0.0001%, even more preferably at least 0.001%, still even more preferably at least 0.01%, and most preferably at least 0.1% by weight of the composition. Preferably, the carboxymethyl cysteine compound is present in amount of no greater than 10%, more preferably no greater than 5%, even more preferably no greater than 3%, still even more preferably no greater than 1%, and most preferably no greater than 0.5% by weight of the composition.

Preferably, the lysine carboxymethyl cysteinate is present in amount of no greater than 10%, more preferably no greater than 5%, even more preferably no greater than 3%, still even more preferably no greater than 1%, and most preferably no greater than 0.5% by weight of the composition. Preferably, the lysine carboxymethyl cysteinate is present in amount of at least 0.00001%, more preferably at least 0.0001%, even more preferably at least 0.001%, still even more preferably at least 0.01%, and most preferably at least 0.1% by weight of the composition.

The composition of the present invention comprises Sandalwood (Santalum album) extract. Preferably the Sandalwood extract according to the invention is obtained from wood of the Sandalwood. Preferably, the Sandalwood extract comprises extract having INCI (International Nomenclature Cosmetic Ingredient) name of Sandalwood oil and/or Sandalwood wood extract. More preferably, the Sandalwood extract comprises extract having INCI name of Sandalwood wood extract. Most preferably, the Sandalwood extract is extract having INCI name of Sandalwood wood extract.

Preferably, the Sandalwood extract is obtainable by extracting Sandalwood using a supercritical fluid. Preferably the Sandalwood extract is obtainable by extracting a Sandalwood using a supercritical carbon dioxide. Preferably, the Sandalwood extract is obtainable by extracting a Sandalwood wood using a supercritical carbon dioxide. Preferably the extraction is carried out using a fluid in the supercritical state, preferably carbon dioxide and/or xenon, in the presence of a co-solvent chosen from primary or secondary alcohols. Preferably the extraction temperature is 35 to 85° C., more preferably 45 to 75° C., and most preferably 55 to 65° C. The pressure within the extractor is typically 90 to 1000 bar, preferably 150 to 700 bar, and more preferably 250 to 400 bar.

Preferably, the Sandalwood extract is present in amounts of 0.00001 to 5% by weight of the composition, more preferably in amounts of 0.0001 to 1%, even more preferably 0.0005 to 0.2%, still even more preferably 0.001 to 0.1% by weight of the total composition. Preferably the Sandalwood wood extract is present in amounts of 0.00001 to 5% by weight of the composition, more preferably in amounts of 0.0001 to 1%, even more preferably 0.0005 to 0.2%, still even more preferably 0.001 to 0.1% by weight of the total composition.

For sake of clarity, the amount of extract in the present invention refers to the amount of extracted ingredients from the plant, excluding the weight of the extract solvent.

Preferably, the weight ratio of the carboxymethyl cysteine compound to the Sandalwood extract is 2:1 to 5000:1, more preferably 3:1 to 1500:1, even more preferably 10:1 to 600:1 and still even more preferably 40:1 to 150:1. Preferably, the weight ratio of the lysine carboxymethyl cysteinate to the Sandalwood extract is 2:1 to 5000:1, more preferably 3:1 to 1500:1, even more preferably 10:1 to 600:1 and still even more preferably 40:1 to 150:1. Preferably, the weight ratio of the lysine carboxymethyl cysteinate to the Sandalwood wood extract is 2:1 to 5000:1, more preferably 3:1 to 1500:1, even more preferably 10:1 to 600:1 and still even more preferably 40:1 to 150:1.

Preferably, the composition comprises Vitamin B3 compounds (including derivatives of vitamin B3). The vitamin B3 compounds comprises niacin, nicotinic acid, niacinamide or a mixture thereof. The most preferred vitamin B3 compound is niacinamide. Amount of Vitamin B3 compounds may be 0.1 to 10%, preferably 0.5 to 5% by weight of the composition.

Preferably, the composition comprises a glutamate source selected from the group consisting of glutamine, glutamine ester, glutamic acid, pyroglutamic acid, salts, and mixtures thereof. More preferably, the composition comprises pyroglutamic acid and/or salt of pyroglutamic acid. Even more preferably, the composition comprises sodium salt of pyroglutamic acid. Preferably, the glutamate source is present in amount of 0.0001 to 10% by weight of the composition, more preferably 0.001 to 6%, even more preferably 0.01 to 3% by weight of the composition.

The composition may optionally comprise whitening pigment. Whitening pigments are typically particles of high refractive index materials. For example, the whitening pigment may have a refractive index of greater than 1.3, more preferably greater than 1.8 and most preferably from 2.0 to 2.7. Examples of such whitening pigment are those comprising bismuth oxy-chloride, boron nitride, barium sulfate, mica, silica, titanium dioxide, zirconium oxide, aluminium oxide, zinc oxide or combinations thereof. More preferred whitening pigment are particles comprising titanium dioxide, zinc oxide, zirconium oxide, mica, iron oxide or a combination thereof. Even more preferred whitening pigment are particles comprising zinc oxide, zirconium oxide, titanium dioxide or a combination thereof as these materials have especially high refractive index. Still even more preferably the whitening pigment is selected from titanium dioxide, zinc oxide or a mixture thereof and most preferred whitening pigment is titanium dioxide. The average diameter of whitening pigment is typical from 15 nm to 1 micron, more preferably from 35 nm to 800 nm, even more preferably from 50 nm to 500 nm and still even more preferably from 100 to 300 nm. Amount of whitening pigment may be 0.1 to 15%, preferably 0.5 to 5% by weight of the composition.

Preferably, the composition comprises polyhydric alcohol. Polyhydric alcohols may be selected from group of glycerin, propylyene glycol, dipropylene glycol, polypropylene glycol, polyethylene glycol, sorbitol, hydroxypropyl sorbitol, hexylene glycol, 1,3-butylene glycol, isoprene glycol, ethoxylated glycerol, propoxylated glycerol or a mixture thereof. Most preferred polyhydric alcohol is glycerol known also as glycerin. The amount of polyhydric alcohol may range anywhere from 0.1 to 20%, preferably 0.5 to 15% and more preferably 2 and 10% by weight of the composition.

Preferably, the composition comprises emollient materials. Suitable emollient materials include silicones, hydrocarbons, triglycerides or a mixture thereof. These silicones may be organic, silicone-containing or fluorine-containing, volatile or non-volatile, polar or non-polar. Hydrocarbons may include mineral oil, petrolatum and polyalpha-olefins. Examples of preferred volatile hydrocarbons include polydecanes such as isododecane and isodecane (e.g. Permethyl-99A which is available from Presperse Inc.) and the C7-C8 through C12-C15 isoparaffins (such as the Isopar Series available from Exxon Chemicals). Illustrative triglycerides but not limiting are sunflower seed oil, cotton oil, canola oil, soybean oil, castor oil, borage oil, olive oil, shea butter, jojoba oil and mixtures thereof. Mono- and di-glycerides may also be useful. Particularly preferable are glyceryl monostearate and glyceryl distearate.

Preferably, the composition comprises moisturizing agents. Particularly preferred moisturizing agents includes, petrolatum, aquaporin manipulating actives, oat kernel flour, substituted urea like hydroxyethyl urea, hyaluronic acid and/or its precursor N-acetyl glucosamine, hyaluronic acid and/or its precursor N-acetyl glucosamine, or a mixture thereof.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

The composition may comprise water in amount of 10 to 96% by weight of the composition, more preferably from 25 to 92%, even more preferably from 42 to 88%, most preferably from 55 to 82% by weight of the composition.

Preferably, the composition has a viscosity of at least 10 mPa·s, more preferably in the range 30 to 10000 mPa·s, even more preferably 50 to 5000 mPa·s, and most preferably 100 to 2000 mPa·s, when measured at 20 degrees C. at a relatively high shear rate of about 20 s⁻¹. Preferably, the composition is in the form of fluid.

Preferably, the composition is an emulsion, more preferably an oil-in-water emulsion. The skin care composition refers to a composition suitable for topical application to human skin, including leave-on and wash-off products but preferably leave-on compositions. The term “leave-on” as used with reference to compositions herein means a composition that is applied to or rubbed on the skin, and left thereon. The term “wash-off” as used with reference to compositions herein means a skin cleanser that is applied to or rubbed on the skin and rinsed off substantially immediately subsequent to application. Preferably the skin care composition means a fluid liquid, and particularly a moisturizer rather than a make-up product.

Preferably, the personal care composition is a skin care composition. The term “skin” as used herein includes the skin on the face, neck, chest, abdomen, back, arms, under arms, hands, and legs. Preferably “skin” means includes the skin on the face and under arms, more preferably skin means skin on the face other than lips and eyelids.

Preferably the use is non-therapeutic. Preferably the method is non-therapeutic. The term non-therapeutic typically means for cosmetic purposes and not curative or therapeutic purposes.

The following examples are provided to facilitate an understanding of the invention. The examples are not intended to limit the scope of the claims.

### EXAMPLES

### Materials

| | | | | Active |
|---|---|---|---|---|
| Supplier | Trade name | Active | Abbreviation | wt % |
| Sinerga | HAIR APP | Lysine carboxymethyl | LCC | >98% |
| | | cysteinate | | |
| Ashland | Santalwood ™ | Sandalwood extract* | — | 0.5% |

*The sandalwood extract was obtained from Ashland (China) Investing Company. The sandalwood for preparing the Sandalwood extract was obtained in Australia.

### Example 1

This Example demonstrates the improved anti-aging effect by combining lysine carboxymethyl cysteinate and Sandalwood extract.

The normal human epidermal keratinocytes (NHEK) (Biocell, Xi'an, China) were incubated in medium together with or without actives for 24 hours. After incubation, the total RNA for each NHEK was extracted using RNAiso Plus (TaKaRa, Cat: 9108; Cat: RR036) according to manufacturer's protocol.

The extracted RNA was quantified using Nanodrop spectrometer (Thermo Fisher Scientific, Waltham, MA, US) and reverse transcribed to generate the template cDNA using the PrimeScript II 1st Strand cDNA synthesis kit according to manufacturer's protocol. Amplification of the gene encoding the hyaluronan synthase 3 (HAS3) and CD44 was performed with RT-PCR reagent (SYBR® Premix Ex Taq™) on the ABI Vii 7 Real-Time PCR Systems (Applied Biosystems, Thermo fisher scientific, Carlsbad, CA, USA). The beta-actin gene was selected as the housekeeping gene and all data on relative expression of the target genes was normalized to beta-actin. The fold changes in expression were calculated relative to the blank control (medium having no active). All tests were conducted at least three times and Table 1 shows the fold changes in expression of HAS3 and CD44.

TABLE 1

| | | HAS3 | CD44 |
|---|---|---|---|
| NHEK | Incubated in ^{#} | expression | expression |
| 1(Blank | Medium | 1.02 | 1.03 |
| control) | | | |
| 2 | 0.02 wt % of Santalwood ™ in | 1.87 | 2.45 |
| | medium | | |
| 3 | 0.05 wt % of LCC in medium | 0.84 | 1.06 |
| 4 | 0.02 wt % of Santalwood ™ and | 3.2 * | 3.14 * |
| | 0.05 wt % of LCC in medium | | |

^{#} The level of the active is based on the medium.
* Significantly elevated (p < 0.05) than any of single active.

As demonstrated in Table 1, it was surprisingly found that both HASS expression and CD44 expression were significantly elevated by combining lysine carboxymethyl cysteinate and Sandalwood extract.

1. A personal care composition comprising carboxymethyl cysteine compound and Sandalwood extract.

2. The personal care composition according to claim 1, wherein the carboxymethyl cysteine compound comprises carboxymethyl cysteine, ester of carboxymethyl cysteine, and/or salt of carboxymethyl cysteine.

3. The personal care composition according to claim 2, wherein the carboxymethyl cysteine compound comprises a salt of carboxymethyl cysteine.

4. The personal care composition according to claim 1, wherein the carboxymethyl cysteine compound is present in an amount of at least 0.00001% by weight of the composition and no greater than 10% by weight of the composition.

5. The personal care composition according to claim 1, wherein the Sandalwood extract comprises Sandalwood oil and/or Sandalwood wood extract.

6. The personal care composition according to claim 1, wherein the Sandalwood extract is obtainable by extracting Sandalwood using a supercritical fluid.

7. The personal care composition according to claim 1, wherein the Sandalwood extract is present in amounts of 0.00001 to 5% by weight of the composition.

8. The personal care composition according to claim 1, wherein the carboxymethyl cysteine compound to the Sandalwood extract has a weight ratio of 2:1 to 5000:1.

9. The personal care composition according to claim 1, wherein the lysine carboxymethyl cysteinate to the Sandalwood extract has a weight ratio of 2:1 to 5000:1.

10. The personal care composition according to claim 1, wherein the composition is an oil-in-water emulsion.

11. The personal care composition according to claim 1, wherein the composition comprises water in an amount of 10 to 96% by weight of the composition.

12. A method of improving skin moisturization; treating, reducing, and/or preventing of fine lines or wrinkles of skin; improving collagen production in skin; providing anti-aging benefit to skin; and/or firming skin, comprising a step of topically applying to the skin the personal care composition according to claim 1.

13. (canceled)

14. The personal care composition according to claim 2, wherein the carboxymethyl cysteine compound comprises lysine carboxymethyl cysteinate.

15. The personal care composition according to claim 1, wherein the carboxymethyl cysteine compound is present in an amount of at least 0.01% by weight of the composition and no greater than 3% by weight of the composition.

16. The personal care composition according to claim 1, wherein the Sandalwood extract comprises extract is Sandalwood wood extract.

17. The personal care composition according to claim 1, wherein the Sandalwood extract is obtainable by extracting Sandalwood using supercritical carbon dioxide.

18. The personal care composition according to claim 1, wherein the Sandalwood extract is present in amounts of 0.001 to 0.1% by weight of the total composition.

19. The personal care composition according to claim 1, wherein the carboxymethyl cysteine compound to the Sandalwood extract has a weight ratio of 10:1 to 600:1.

20. The personal care composition according to claim 1, wherein the carboxymethyl cysteine compound to the Sandalwood extract has a weight ratio of 40:1 to 150:1.

21. The personal care composition according to claim 1, wherein the lysine carboxymethyl cysteinate to the Sandalwood extract has a weight ratio of 40:1 to 150:1.
